(19) United States
(12) Patent Application Publication
(10) Pub. No.: US 2011/0257747 A1
(43) Pub. Date: Oct. 20, 2011

(54) KIT FOR CONSTRUCTING A SPINAL DISK PROSTHESIS, AND SYSTEM FOR CONSTRUCTING DIFFERENT SPINAL DISK PROSTHESES

(76) Inventor: Franz Copf, Stuttgart (DE)

(21) Appl. No.: 13/120,584

(22) PCT Filed: Sep. 24, 2009
(86) PCT No.: PCT/EP09/06906

§ 371 Date: Jun. 29, 2011

(30) Foreign Application Priority Data
Sep. 24, 2008 (DE) 10 2008 048 739.2

(51) Int. Cl. A61F 2/44

(52) U.S. Cl. 623/17.15

(57) ABSTRACT

The invention relates to a kit for constructing a spinal disk prosthesis provided for arrangement in a spinal disk compartment limited by vertebral bodies, comprising two prosthesis plates (10) which are disposed in a manner movable relative to each other, the exterior surfaces (12) of which are provided for contacting surfaces of vertebral bodies positioned on the opposite side, and the interior surfaces (14) of which form corresponding bearing surfaces of a pivot bearing (34; 134; 334; 434) after assembly. According to the invention, the kit further comprises a limiting device (24, 26; 150, 152; 254, 256; 364; 466), which is associated with the pivot bearing (34; 134; 334; 434), for limiting a pivot movement about a rotational axis (36) oriented substantially normally to the exterior surfaces (12) of the prosthesis plate (10). For this purpose, the limiting device may be connected to the prosthesis plates (10).

### BACKGROUND OF THE INVENTION

1. Field of the Invention

The invention relates to a kit for constructing a spinal disc prosthesis provided for arrangement in a spinal disc compartment limited by vertebral bodies. The invention furthermore relates to a system for constructing differently configured spinal disc prostheses of this type.

2. Description of the Prior Art

Spinal disc prostheses are implanted when damage to a spinal disc necessitates replacement of the damaged spinal disc. Here, the spinal disc prosthesis is inserted into a spinal disc compartment of the vertebral column limited by two adjacently arranged vertebral bodies.

Spinal disc prostheses are intended to ensure that the mobility of the patient is impaired as little as possible and are frequently constructed as prosthesis plates connected to one another in articulated manner. To enable advantageous adaptation of the spinal disc prosthesis to the anatomical properties, it is known from WO 2007/003438 A2 to provide spinal disc prostheses in different configurations. The configurations differ, for example, in terms of the size of the prosthesis plates, the spacing between the prosthesis plates and the position of the centre of movement, i.e. centre of rotation, about which the prosthesis plates can be pivoted relative to one another. To this end, the spinal disc prosthesis has intermediate elements which can be constructed as joint elements or pivot angle limiters. For this purpose, receiving shafts, which are constructed to receive the intermediate elements, are provided in the prosthesis plates. By means of the replaceable intermediate elements, it is possible to set the spacing and the maximum pivot angle between the prosthesis plates and specify the position of the centre of movement.

A pivot bearing of the known spinal disc prosthesis is constructed as a ball and socket joint and, in the implanted state in the spinal disc compartment, has three rotational degrees of freedom of movement. The movement properties of the smallest functional unit of the spinal column (also known as the Junghans Movement Segment or Functional Spinal Unit (FSU)), which is originally formed by the opposing vertebral bodies and the spinal disc, should be reproduced as far as possible by the spinal disc prosthesis. Like the original spinal disc, the spinal disc prosthesis should enable forward bending (also known as flexion), backward stretching (also known as extension), sideways bending to the right or left (also as lateral flexion), and a rotational movement which is effected substantially about a rotational axis extending along the vertebral column and is also known as rotation.

The natural spinal disc comprising an external, flexible connective tissue ring and a soft core surrounded by this latter serves as a flexible joint and damping element and is deformed upon relative movements of the vertebral bodies. In addition to its function as a joint element, the construction of the spinal disc also means that it functions as a deflection limiter for the relative movements of the vertebral bodies. From the above-mentioned WO 2007/003438 A2, it is known to arrange replaceable stops on opposing prosthesis plates, which stops enable a limitation of the pivotal movement of the prosthesis plates upon sideways bending (lateral flexion) to the right or left.

US 2008/0221693 A1 discloses a spinal disc prosthesis in which the joint elements forming a ball and socket joint are constructed in one piece on the prosthesis plates. Protrusions formed on a prosthesis plate in the region of a spherical cap here engage in cavities which are formed in the other prosthesis plate in the region of a hollow spherical cup complementary to the spherical cap. With the cavities, the protrusions form a limiting device which serves to limit a rotational movement about a rotational axis aligned substantially normally to the exterior surfaces of the prosthesis plates. However, the maximum permitted rotational angle in this known spinal disc prosthesis cannot be altered.

US 2005/0234553 A1 discloses a spinal disc prosthesis which has a flexible, overall cup-shaped body, the wall of which is provided with a helically circumferential slot. A first joint element, which forms a ball and socket joint with a second joint element, is fixed at the base of the cup-shaped body. The second joint element here is in turn fixed circumferentially at the top of the inside edge of the cup-shaped body. The body fixedly connected to the joint elements provides torsional resistance to a relative rotation of the joint elements about an axis extending perpendicularly to the cup base, which torsional resistance results in a limitation of the rotational movement. However, the very simple construction of this known spinal disc prosthesis also means that this limiting device for the rotational movement cannot be adjusted irrespective of the dimensioning of the spinal disc prosthesis.

### SUMMARY OF THE INVENTION

The object of the invention is to demonstrate a kit for constructing a spinal disc prosthesis by means of which it is possible to set the maximum permissible rotational angle irrespective of the configuration of the prosthesis plates, the dimensioning of which should ideally be dependent on many different factors.

This object is achieved by a kit having
a) two prosthesis plates which are arranged such that they are movable relative to one another, the exterior surfaces of which are provided for contacting surfaces of opposing vertebral bodies and the interior surfaces of which form corresponding bearing surfaces of a pivot bearing after assembly, and having
b) a limiting device, associated with the pivot bearing, for limiting a rotational movement about a rotational axis aligned substantially normally to the exterior surfaces of the prosthesis plates, wherein the limiting device may be connected to the prosthesis plates.

The inventor has recognised that the maximum permissible rotational angle can only be specified irrespective of the construction of the prosthesis plates when the limiting device for limiting the rotational movement is constructed as an independent component which may be connected to the prosthesis plates. This considerably reduces the number of components to be included when aiming to provide spinal disc prostheses which, whilst permitting different maximum rotational angles, can also have differently configured prosthesis plates. If, for example, the aim is to enable a choice of four different maximum rotational angles and four different geometries for prosthesis plates, according to the invention it is sufficient to provide four different limiting devices and four different sets of prosthesis plates. With the prior art, on the other hand, in which the limitation of the rotational angle is always an integral part of the prosthesis plates, it is necessary to provide a total of 16 different sets of such parts to achieve the same options. This increases the cost of maintaining and disinfecting the prostheses.

After the implantation of the spinal disc prosthesis in the spinal disc compartment, the exterior surfaces of the prosthesis plates lie substantially flat against the mutually facing surfaces of adjacent vertebral bodies. The surfaces of the vertebral bodies are aligned substantially parallel to one another in a neutral position of the vertebral column. Moreover, the surfaces of the vertebral bodies in the neutral position of the vertebral column are aligned normally to a portion of a centre axis of the vertebral column which, in a view from the side of the vertebral column, curves in an S-shape and, in a view from the rear of the vertebral column, is substantially linear. With a rotational movement of the vertebral column out of the neutral position, adjacent vertebral bodies are twisted relative to one another substantially about the centre axis of the vertebral column. As a result of this, upon a rotational movement of this type, the prosthesis plates are moved relative to one another in a rotational plane which is aligned substantially normally to the centre axis of the vertebral column. The centre axis of the vertebral column thus corresponds substantially to the rotational axis of the prosthesis plates.

The kit according to the invention enables a system for constructing different spinal disc prostheses to be realised, which has:
a) a set of differently configured pairs of prosthesis plates, which may be arranged such that they are movable relative to one another, the exterior surfaces of which are provided for contacting surfaces of opposing vertebral bodies and the interior surfaces of which form corresponding bearing surfaces of a pivot bearing after assembly, and
b) a set of limiting devices, associated with the pivot bearing, for limiting a rotational movement of the prosthesis plates about a rotational axis aligned substantially normally to the exterior surfaces of the prosthesis plates, wherein the limiting devices of the set differ from one another at least in that they limit the rotational movement to different maximum rotational angles, and wherein the limiting devices may be connected to different prosthesis plates.

In principle, it is possible to connect the limiting device permanently to the prosthesis plates. For example, a latching mechanism which is no longer readily detachable or even a force-fitting connection, perhaps by adhesion, is possible. In a preferred construction of the invention, however, the limiting device may be detachably connected to the prosthesis plates. On the one hand, this is advantageous in that, if a limiting device is connected to the prosthesis plates by mistake, this does not mean that the assembly thereby produced can no longer be used at least for the patient in question. On the other hand, a detachable connection with a suitable design of the limiting device still enables this latter to be replaced with another limiting device if necessary even after the spinal disc prosthesis has been implanted in the spinal disc compartment. Such a requirement can arise, for example, when a too-generously calculated maximum rotational angle has resulted in the adjoining segments of the vertebral column becoming overloaded. In this case, it is possible during an operation to release the connection, remove the limiting device and replace it with a limiting device which only enables a relatively small maximum rotational angle.

In another exemplary embodiment, the limiting device or a part thereof is connected to an insertion element which may be introduced into a receiving shaft constructed in one of the prosthesis plates. The receiving shaft here can be adapted to the insertion element in such a way that the insertion element may only be introduced into the receiving shaft in one direction.

The surgeon is thus able to assemble the spinal disc prosthesis very simply from the prosthesis plates and the limiting device, even during the operation. This in turn enables the choice of limiting device from factors make possible which the surgeon is only able to calculate or otherwise establish during the operation.

In another exemplary embodiment, the limiting device comprises at least one flexible connecting element which extends between the prosthesis plates and by means of which the rotational movement about the rotational axis may be limited without the assistance of stops. The flexible connecting element is preferably arranged without tension in a neutral position of the prosthesis plates. By twisting or pivoting the prosthesis plates relative to one another, the fixing points for the connecting element on the prosthesis plates can be distanced from one another so that the connecting element is under tension. This causes the rotational movement to end when a balance of forces is reached between the torque introduced onto the vertebral bodies and the restoring moment caused by the tensile stress produced in the limiting device.

To ensure a rotational-angle dependent braking effect by means of the at least one connecting element, the connecting element is preferably of an elastic construction. The connecting element can be made for example from a woven fabric, knitted fabrics or a mesh fabric, in which fibres of a material with a low elasticity, for example carbon fibres or titanium wires, are arranged relative to one another in such a way that the internal friction between the fibres results in the desired braking effect with the introduction of a tensile force. Alternatively, the connecting element can be manufactured as a woven fabric, knitted fabrics or a mesh fabric from elastic fibres such as aramide fibres or super-elastic wires made from nickel-titanium alloys.

The connecting element can also be manufactured in one piece from an elastic material. This enables a single-piece construction of the connecting element in the manner of a round cord or a block which is preferably provided with a profile defined by cavities to ensure a high degree of elasticity. Fluoroelastomers, particularly perfluoro rubber, tetrafluoroethylene/propylene rubbers and fluorinated silicone rubber can be used as materials for a connecting element of this type.

In another exemplary embodiment, the connecting element is connected to two insertion elements which may be introduced into receiving shafts constructed on the two prosthesis plates. Particularly simple assembly of the spinal disc prosthesis is also enabled here by the use of insertion elements and receiving shafts adapted thereto.

In another exemplary embodiment, the limiting device comprises braking surfaces which are aligned in such a way that, with a rotation of a prosthesis plate about the rotational axis, a braking surface which is arranged on one of the two prosthesis plates slides on a corresponding braking surface which is arranged on the other prosthesis plate, wherein the frictional resistance between the braking surfaces increases with the increasing rotational angle as a result of the pressure exerted by the vertebral bodies.

The braking surfaces are preferably spaced from one another in a neutral position of the spinal disc prosthesis so as to enable sideways bending (lateral flexion) in a predetermined pivot angle range.

In a further development of this construction, the braking surfaces are constructed in such a way that the spacing between the prosthesis plates increases with the increasing rotational angle. With an increase in the spacing of the prosthesis plates, the ligaments arranged around the vertebral column are stretched and result in an additional increase in the pressure exerted by the vertebral bodies. The frictional resistance between the braking surfaces is thereby also increased. Using the anatomical properties of the patient, a braking action counteracting the rotation is thus achieved, and this increases strongly, but always steadily, with the increasing rotational angle and thus prevents the temporary load peaks which perhaps occur when impacts are not damped.

The braking surfaces here can be aligned in such a way that the braking surface on one prosthesis plate slides on the corresponding braking surface on the other prosthesis plate only after a predetermined rotational angle has been exceeded. In this case, the braking action is therefore only initiated from a certain rotational angle. This behaviour corresponds to that of the natural spinal disc prosthesis since, when the rotational angle is small, this spinal disc also only exerts an extremely low resistance against the rotation.

It is favourable if the braking surfaces are not part of the pivot bearing. This ensures that any friction or other wear of the braking surfaces cannot have a detrimental effect on the properties of the pivot bearing.

In another exemplary embodiment, the limiting device comprises at least two stop surfaces, associated with opposing prosthesis plates, for limiting the rotational angle. It is thus possible to realise a particularly simple construction of the spinal disc prosthesis.

In a further development of the invention, it is arranged for the limiting device to be set to provide a rotational-angle dependent braking force in a rotational-angle range which accounts for at least 10 percent, preferably at least 20 percent, of a predeterminable rotational-angle range of the prosthesis plates. The maximum rotational angle is predetermined structurally by the design of the spinal disc prosthesis. The maximum rotational angle is the angle covered by a rotation of the implanted prosthesis plates from a neutral position in one of the two rotational directions until there is a complete braking of the rotational movement between the prosthesis plates as a result of the braking force or braking moment. The limiting device according to the invention provides the braking force or the braking moment in addition to a frictional force which is produced in any case by friction between the joint surfaces of the prosthesis plates. According to the invention, the braking force is provided when the rotational angle between the prosthesis plates has reached a value which corresponds to at least 90 percent, preferably at least 80 percent, of the maximum rotational angle.

It is advantageous if the limiting device is arranged in such a way that the maximum rotational angle and/or the braking force provided for the rotational movement are related to a pivot angle formed between the prosthesis plates by way of a characteristic curve which extends preferably continuously. A pivotal movement of the prosthesis plates is effected about pivot axes of the pivot bearing which are located at least approximately in the rotational plane of the rotational movement. Pivotal movements of the prosthesis plates occur during the flexion, during the extension and during the lateral flexion of the vertebral column. With a pivotal movement of the prosthesis plates about at least one pivot axis, a stretching of the ligaments, muscles and nerves surrounding the vertebral column already takes place. If the pivotal movement is superimposed by a rotational movement, further stretching of the ligaments, muscles and nerves takes place. Since the extension should not exceed a predetermined value, the limiting device ensures that the maximum rotational angle is reduced as the pivot angle(s) increase(s). This characteristic of the spinal disc prosthesis reproduces the behaviour of the natural spinal disc in which the torsional stresses produced by twisting the vertebral bodies and the spinal disc arranged between them are superimposed over the compressive stresses in the spinal disc caused by the pivotal movement and thus result in a greater movement resistance.

### BRIEF DESCRIPTION OF THE DRAWINGS

Exemplary embodiments of the invention are illustrated in the drawings, which show:

FIG. 1 a plan view of a prosthesis plate with replaceable stop elements and a replaceable joint recess;

FIG. 2 a side view of the prosthesis plate according to FIG. 1;

FIG. 3 a plan view of a prosthesis plate with replaceable stop elements and a replaceable spherical joint head;

FIG. 4 a side view of the prosthesis plate according to FIG. 3;

FIG. 5 a spinal disc prosthesis formed by the prosthesis plates according to FIGS. 1 and 3;

FIG. 6 a plan view of a prosthesis plate with replaceable stop elements and a replaceable spherical joint head which has two stop projections;

FIG. 7 a sectional illustration of the prosthesis plate according to FIG. 6;

FIG. 8 a plan view of a prosthesis plate with replaceable stop elements and a replaceable joint recess which is provided with movement spaces for stop projections;

FIG. 9 a sectional illustration of the prosthesis plate according to FIG. 8;

FIG. 10 a spinal disc prosthesis formed by the prosthesis plates according to FIGS. 6 and 8;

FIG. 11 a plan view of a prosthesis plate with a replaceable joint recess which is provided with an elastic locking web;

FIG. 12 a sectional illustration of the prosthesis plate according to FIG. 11;

FIG. 13 a plan view of a prosthesis plate with a replaceable spherical joint head which is provided with a movement groove;

FIG. 14 a sectional illustration of the prosthesis plate according to FIG. 13;

FIG. 15 a spinal disc prosthesis with flexible connecting elements which are mounted on insertion elements for the prosthesis plates; and

FIG. 16 a spinal disc prosthesis with a connecting element mounted between the prosthesis plates on insertion elements.

In all the Figures, functionally identical components are provided with the same reference numerals.

### DESCRIPTION OF PREFERRED EXEMPLARY EMBODIMENTS

The exemplary embodiments described below for spinal disc prostheses each comprise prosthesis plates 10 which are made from a metal material and have a reniform outer contour. An exterior surface 12 of the prosthesis plates 10 serves to contact a vertebral body (not illustrated) and is provided with a bulge 16 which engages in a ring of the vertebral body, which is formed from a more solid bone material. An interior surface 14 of the prosthesis plates 10 has three shafts 18, 20, 22 which are arranged to receive insertion elements. Other constructions of prosthesis plates can likewise be used to realise the features according to the invention.

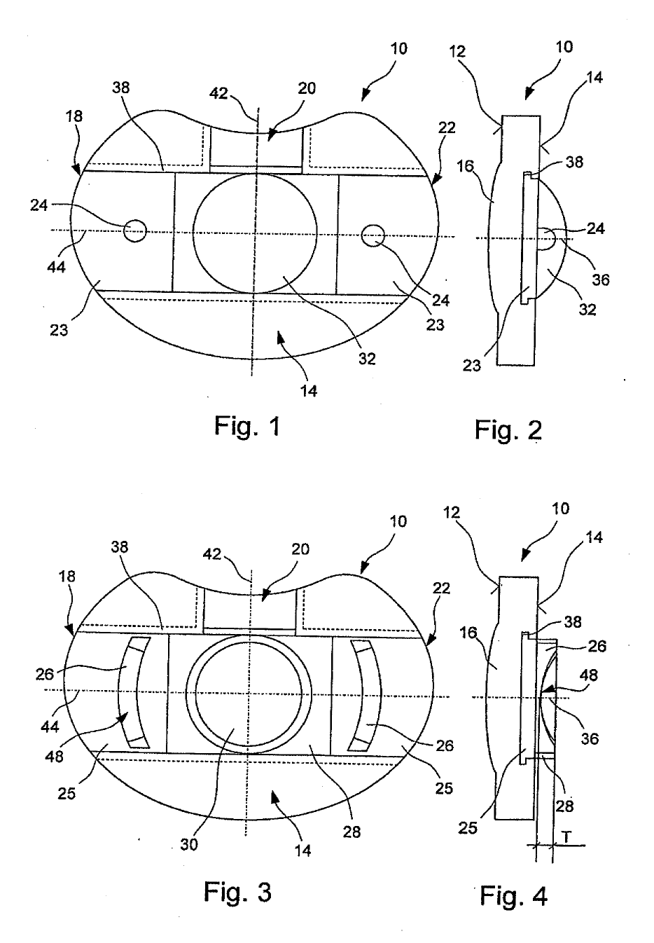

In the prosthesis plate 10 according to FIGS. 1 and 2, insertion elements 23 provided with braking pins 24 are received in the shafts 18 and 22. In the prosthesis plate 10 according to FIGS. 3 and 4, insertion elements 25 provided with braking ramps 26 are received in the shafts 18 and 22.

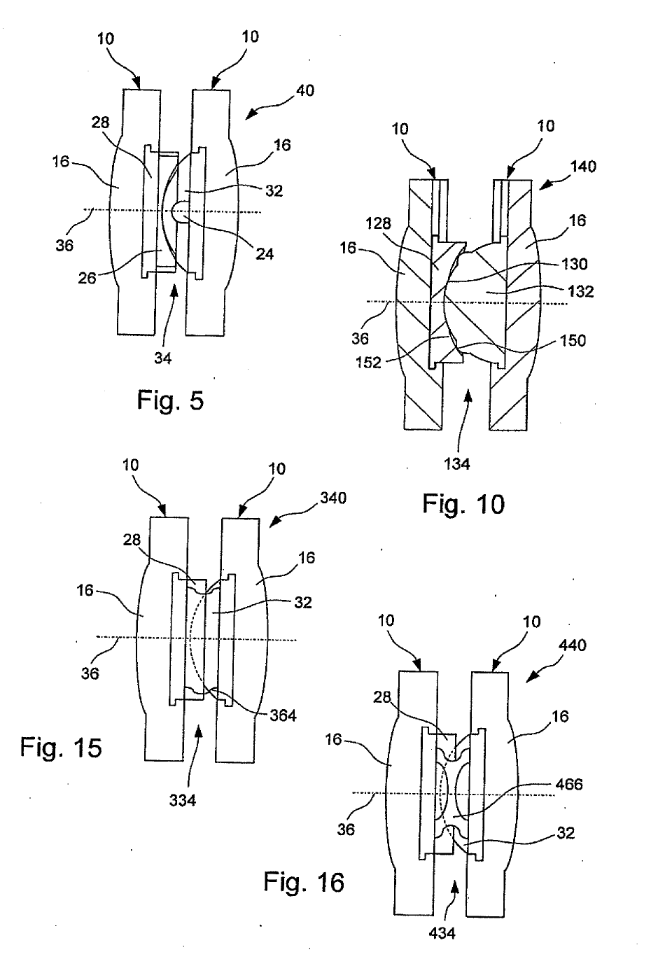

In the prosthesis plate 10 according to FIGS. 3 and 4, a joint plate 28, which is provided with a concavely formed, spherical cap-shaped joint recess 30, is received in the shaft 20. The joint recess 30 forms a ball and socket joint 34 (illustrated in more detail in FIG. 5) with the spherical joint head 32 arranged in the prosthesis plate 10 according to FIGS. 1 and 2. The ball and socket joint 34 enables a rotational movement of the prosthesis plates 10 about the rotational axis 36 which, in the neutral position shown, is aligned normally to the exterior surfaces 12 of the prosthesis plates 10, and pivotal movements of the prosthesis plates 10 about pivot axes 42 and 44, as shown by way of example in FIGS. 1 and 3. In the neutral position of the spinal disc prosthesis 40 shown in FIG. 5, the pivot axes 42 and 44 are located in a plane aligned orthogonally to the rotational axis 36. Thus, the ball and socket joint 34 enables a relative movement of the prosthesis plates 10 of the spinal disc prosthesis 40 with three rotational degrees of freedom.

To ensure that the insertions elements 23, 25 are reliably received in the shafts 18, 20, 22, grooves 38 are provided there in respective edge regions which have a rectangular cross-section and enable the insertion elements 23, 25 to be fixed in the manner of a tongue and groove connection. The cross-section of the groove 38 can be seen for example in FIG. 2. Additional measures for fixing the insertion elements 23, 25 can consist in the choice of a suitable material for achieving a high degree of friction between the insertion element and prosthesis plate 10. It is additionally or alternatively possible to use fastening means (not illustrated) such as screws to reliably fix the insertion elements 23, 25 to the prosthesis plates 10.

A limitation of the pivot angle for pivotal movements of the prosthesis plates 10 is already ensured by their external form of which the interior surfaces 14 move towards one another in certain areas upon the execution of a pivotal movement and come into contact with one another so that further pivoting of the prosthesis plates 10 relative to one another is prevented.

On the other hand, a limitation of the rotational movement of the prosthesis plates about the rotational axis 36 does not take place solely as a result of the form of the prosthesis plates 10. Therefore, the insertion elements 23, 25 equipped with braking pins 24 and braking ramps 26 serve to limit the rotational movement. The braking pin 24 has a spherical cap-shaped form at its end face. The braking ramp 26 is constructed as a curved web with a concave trough-like depression 48. The depth T of the depression 48 and the size of the braking pin 24 are selected such that, in the neutral position according to FIG. 5, both a pivotal movement of the prosthesis plates 10 about the pivot axis 42 (lateral flexion) and a rotation about the rotational axis 36 can take place. With regard to a pivotal movement about the pivot axis 44, which corresponds to a flexion or extension of the vertebral column, the arrangement comprising braking pin 24 and braking ramp 26 is invariant.

As revealed in FIG. 5, upon a relative twisting of the prosthesis plates 10 about the rotational axis 36, the braking pins 24 come into contact with the braking ramps 26 and, with a continued rotational movement, slide onto the regions of the braking ramp 26 which rise with an increasing slope. Undesired tilting of the prosthesis plates 10 is prevented as a result of the asymmetrical construction and arrangement of the braking pins 24 and the braking ramps 26 relative to the pivot axis 42. Depending on the compressive forces introduced onto the exterior surfaces of the prosthesis plates 10 by the vertebral bodies, the sliding procedure of the braking pins 24 on the braking ramps 27 produces frictional forces which increase steadily with the increasing rotational angle and result in a braking of the rotational movement. In relation to the rotational axis 36, the frictional forces result in a braking moment which is introduced onto the vertebral bodies by way of the prosthesis plates 10 and which opposes the torque introduced by the ligaments or by external forces.

As a result of the trough-shaped design of the depressions 48, an increase in the spacing of the prosthesis plates 10 can take place with a further increase of the rotational angle since the braking pins 24 slide onto the rising regions of the braking ramps 26.

Thus, the ligaments arranged around the vertebral column are additionally strained, resulting in an increase in the compressive forces on the prosthesis plates 10 and the resultant frictional forces. The braking moment produced by the frictional forces thus increases with the increasing rotational angle.

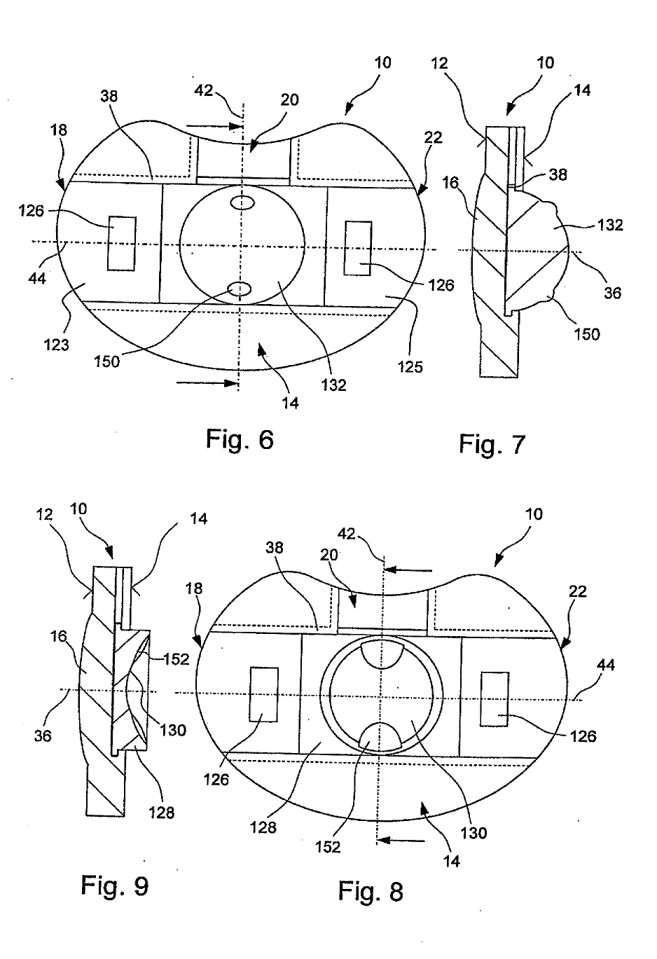

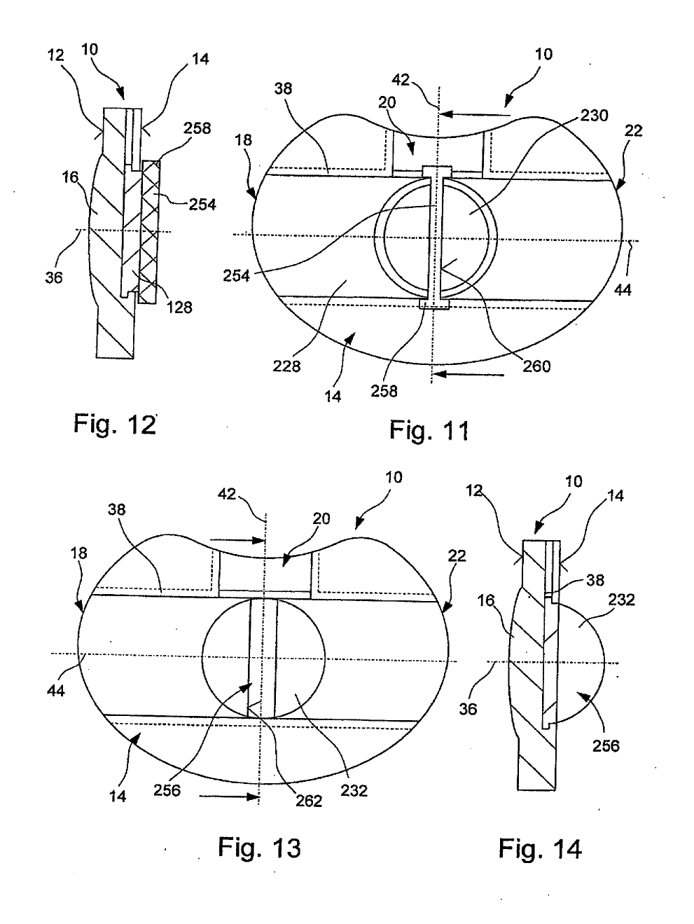

In the exemplary embodiment of a spinal disc prosthesis 140 illustrated in FIGS. 6 to 10, the insertion elements 123, 125 are provided with square stops 126 which serve to limit a pivotal movement (lateral flexion) about the pivot axis 42. To limit the rotational angle, two projections 150 constructed in the shape of a spherical cap are mounted on the spherical joint head 132. Control recesses 152 corresponding to the projections 150 are provided on the joint recess 130 and serve to limit the rotational angle. As shown in FIG. 10, the control recesses 152 are constructed as curved cavities in the joint recess 130 and thus enable both a limitation of a pivotal movement and also a limitation of a rotational movement of the prosthesis plates 10. The control recesses 152 here are constructed in such a way that a relative pivotal movement of the prosthesis plates 10 about the pivot axis 44 is not limited. On the other hand, a relative pivotal movement of the prosthesis plates 10 about the pivot axis 42 is also limited without the square stops 126 illustrated in FIGS. 6 and 8 as a result of the control recesses 152 rising as far as the surface of the joint recess 130. The rotational movement of the prosthesis plates 10 relative to one another is likewise limited as a result of the control recesses 152 rising as far as the surface of the joint recess 130. As the projections 150 approach the edge regions of the control recesses 152, the spacing of the prosthesis plates 10 increases in a manner similar to that in the embodiment according to FIGS. 1 to 5, so that the desired braking action for the rotational movement can thus take place. As a result of the design of the contour of the projections 150 and/or the control recesses 152, it is possible to influence the characteristic of the rotational-angle limitation and possibly the characteristic of the pivot angle limitation. To influence these characteristics, the size of the projections 150 and/or the extent of the control recesses 152 and their curve progression can be varied.

Deviating from the embodiments described above, in the exemplary embodiment of a spinal disc prosthesis 240 according to FIGS. 12 to 14, there is no provision for increasing the spacing of the prosthesis plates 10 for the braking action during the rotational movement. Instead, a web 254 of an elastic plastics material is arranged in the joint recess 230 and is provided for engagement in a slot 256 provided in the spherical joint head 232. The web 254 is provided with thickened portions 258 at its end, which are provided for form-fitting locking of the web 254 in the joint recess 240 provided with corresponding cavities. During a relative pivotal movement of the prosthesis plates 10 about the pivot axis 44, there is no contact between the interior surfaces 262 of the slot 256 and the exterior surfaces 260 of the web 254. During a relative pivotal movement of the prosthesis plates 10 about the pivot axis 42, depending on the dimensioning of the web 254 and the slot 256, contact can occur between opposing surfaces 260, 262, resulting in a limitation of the pivotal angle. During a rotation about the rotational axis 36, the interior surfaces 262 of the slot 256 come into contact with the exterior surfaces 260 of the web 254 when a rotational angle specified by the dimensioning of the web 254 and the slot 256 is exceeded. A transfer of force between the interior surfaces 262 and the exterior surfaces 260 takes place here, which results in a deformation of the elastic web 254. Thus, a restoring force is exerted on the interior surfaces 262, which produces a braking moment on the prosthesis plate 10. In addition to the dimensioning of the web 254, the restoring force also depends on its material properties, particularly its modulus of elasticity. By replacing the web 25, it is possible to make an adjustment to the desired pivot angle and the braking characteristic for the rotational movement.

In the spinal disc prosthesis 340 illustrated in FIG. 15, the insertion elements 323, 325 are provided with pliable fabric hose portions 364 which are made from titanium fibres and whereof the length is dimensioned such that the pivot angle is not limited, even in the event of superimposed pivotal movements about both pivot axes 42, 44. However, if there is a rotational movement of the prosthesis plates 10 relative to one another, the rotational angle is limited by the length of the fabric hose portions 364. The elastic properties of the fabric hose portions 364 enable gentle braking of a rotational movement, they can be set by selecting the aramide fibres accordingly.

In the embodiment of a spinal disc prosthesis 440 illustrated in FIG. 16, a limiting element 466 made from an implantable elastomer, particularly from a fluoroelastomer, is mounted on the opposing insertion elements 468. The limiting element 466 has a high degree of flexibility for pivotal movements about the pivot axes 42 and 44 and is thereby exposed to compressive stresses. Here, the limiting element 466 serves in each case as an elastic stop for pivotal movements about the pivot axes 42, 44. With a relative twisting movement of the opposing prosthesis plates 10, the limiting element 466 is exposed to tensile stresses and limits the rotational angle for the prosthesis plates 10 through its elasticity.

1. A kit for constructing a spinal disc prosthesis provided for arrangement in a spinal disc compartment limited by vertebral bodies, the kit comprising:
(a) two prosthesis plates which are arranged such that they are movable relative to one another, the exterior surfaces of which are provided for contacting surfaces of opposing vertebral bodies and the interior surfaces of which form corresponding bearing surfaces of a pivot bearing after assembly; and
b) a limiting device associated with the pivot bearing, for limiting a rotational movement about a rotational axis aligned substantially normally to the exterior surfaces of the two prosthesis plates, wherein the limiting device can be connected to the two prosthesis plates.

2. A kit according to claim 1, wherein the limiting device can be detachably connected to the two prosthesis plates.

3. A kit according to claim 1, wherein the limiting device or a part thereof is connected to an insertion element which can be introduced into a receiving shaft constructed in one of the two prosthesis plates.

4. A kit according to claim 3, wherein the receiving shaft is adapted to the insertion element in such a way that the insertion element can only be introduced into the receiving shaft in one direction.

5. A kit according to claim 1, wherein the limiting device is arranged in such a way that the maximum rotational angle, the braking force provided for the rotational movement, or both, are related to a pivot angle of the two prosthesis plates by way of a characteristic curve.

6. A kit according to claim 1, wherein the limiting device comprises at least one flexible connecting element which extends between the two prosthesis plates and by means of which the rotational movement about the rotational axis can be limited without the assistance of stops.

7. A kit according to claim 6, the connecting element is manufactured from an elastic material.

8. A kit according to claim 3, wherein the limiting device has at least one flexible connecting element extending between the two prosthesis plates whereby the rotational movement about the rotational axis can be limited without the assistance of stops, and wherein the connecting element is connected to two insertion elements which can be introduced into receiving shafts constructed on the two prosthesis plates.

9. A kit according to claim 1, wherein the limiting device comprises braking surfaces which are aligned in such a way that, with a rotation of one of the two prosthesis plates about the rotational axis, a braking surface which is arranged on one of the two prosthesis plates slides on a corresponding braking surface which is arranged on the other of the two prosthesis plates, wherein the frictional resistance between the braking surfaces increases with the increasing rotational angle as a result of the pressure exerted by the vertebral bodies.

10. A kit according to claim 9, wherein the braking surfaces are constructed in such a way that the spacing between the two prosthesis plates increases with the increasing rotational angle.

11. A kit according to claim 9, wherein the braking surfaces are aligned in such a way that the braking surface on one of the two prosthesis plates slides on the corresponding braking surface on the other of the two prosthesis plates only after a predetermined rotational angle has been exceeded.

12. A kit according to claim 9, wherein the braking surfaces are not part of the pivot bearing.

13. A kit according to claim 1, wherein the limiting device comprises at least two stop surfaces associated with the two prosthesis plates for limiting the rotational angle.

14. A system for constructing different spinal disc prostheses provided for arrangement in a spinal disc compartment limited by vertebral bodies, the system comprising:
a) a set of differently configured pairs of prosthesis plates, which may be arranged such that they are movable relative to one another, the exterior surfaces of which are provided for contacting surfaces of opposing vertebral bodies and the interior surfaces of which form corresponding bearing surfaces of a pivot bearing after assembly; and
b) a set of limiting devices associated with the pivot bearing for limiting a rotational movement of the prosthesis plates about a rotational axis aligned substantially normally to the exterior surfaces of the prosthesis plates, wherein the limiting devices of the set differ from one another at least in that they limit the rotational movement to different maximum rotational angles, and wherein the limiting devices can be connected to different prosthesis plates

15. A kit according to claim 5, wherein the characteristic curve extends continuously.
